(19) United States
(12) Patent Application Publication
(10) Pub. No.: US 2012/0121645 A1
(43) Pub. Date: May 17, 2012

(54) SUPRALINGUAL VACCINES AND APPLICATORS

(75) Inventor: Sanjay Kapil, Stillwater, OK (US)

(73) Assignee: THE BOARD OF REGENTS FOR OKLAHOMA STATE UNIVERSITY, Stillwater, OK (US)

(21) Appl. No.: 13/384,505

(22) PCT Filed: Jul. 15, 2010
(86) PCT No.: PCT/US2010/042142

§ 371 Date: Jan. 17, 2012

Related U.S. Application Data
(60) Provisional application No. 61/226,459, filed on Jul. 17, 2009.

(51) Int. Cl. A61K 39/23; A61K 39/12; A61K 39/135; A61J 7/00; A61P 37/04; A61P 31/20; A61P 31/14; A61K 39/00; A61K 39/125

(52) U.S. Cl. 424/216.1; 424/184.1; 424/204.1; 424/233.1; 604/77

(57) ABSTRACT

Solid and semi-solid formulations are used for supralingual administration of vaccines to animals. The formulations, which comprise antigens dispersed in a solid or semi-solid matrix, or paste, are delivered via supralingual applicators. The supralingual applicators are designed so as to position the antigen-containing matrix directly on the dorsal surface of the tongue during vaccine delivery. Upon exposure to saliva and to suckling and/or licking action of the tongue, the matrix dissolves and releases antigens to the tongue. In some embodiments, the antigens are viruses, for example, attenuated viruses that are capable of infecting cells of the tongue, e.g. canine parvoviruses which infect basal tongue cells. The supralingual applicators are especially useful for the delivery of vaccines to newborn animals.

### FIELD OF THE INVENTION

The invention relates to methods, compositions and devices for administering vaccines to animals, even newborn animals. In particular, the invention provides solid and semi-solid antigen-containing vaccine formulations which are administered using supralingual applicators which are “lollipop-like” or similar in design. Antigens are delivered supralingually by suckling or licking of the applicator by the animal.

### BACKGROUND OF THE INVENTION

Canine parvovirus (CPV) is the number one killer of dogs. Newborn puppies acquire passive immunity against CPV through nursing, especially during the first two days of life, with antibodies in the colostrum of the nursing canine mothers being passed on to the puppy. For many mammals including dogs, the passive immunity provided by the colostrum loses its protective effect sometime around the fifth week of age. Thus, vaccination beginning at 6 weeks is recommended to allow efficacy of the puppy shots. A problem with the current puppy vaccination system, however, is that the early shots (e.g. 3-4 shots given two weeks apart) usually do not immediately elicit an active, protective immune response. A protective immune response may not be present until most or all shots in the series (e.g. at 6, 9, 12 and 15 weeks) have been administered. Unfortunately, the highest mortality (80%) of puppies due to CPV is at 8 weeks of age, when the mother's antibodies are waning and the puppy has not yet established its own active immunity. This window of susceptibility to CPV infection occurs from about 7 to about 11 weeks of age and correlates with high CPV associated mortality.

A simple answer might be to vaccinate puppies earlier. However, this has proven to be problematic because antibodies conferred by the mother also neutralize the CPV in current commercial vaccine preparations, and hence a puppy vaccinated at e.g. 2 or 3 weeks does not elicit an active immune response on its own. The phenomenon of maternal antibody interference is observed in other species as well, such as cats and even humans.

### SUMMARY OF THE INVENTION

The invention provides compositions, devices, methods and protocols to administer vaccines to mammals supralingually i.e. to the surface of the tongue. According to one embodiment of the invention, antigens are delivered or applied to the dorsal surface of the tongue via a supralingual applicator which usually comprises a solid or semi-solid matrix (which may be gelled or solidified in situ) containing one or more antigens of interest. Upon contact with saliva in the tongue, and facilitated by sucking or licking action, the matrix dissolves and releases antigen that coats or partially coats the dorsal surface of the tongue. In some embodiments, the supralingual applicators are used to vaccinate newborn mammals, taking advantage of the innate suckling reflex of the neonate. In certain embodiments the applicators/devices of the invention may be hand-held. In other embodiments, the applicators/devices may be hanging. In some embodiments, the supralingual applicator delivers a virus to the tongue of the animal, and in particular embodiments, the virus can be an attenuated virus that infects basal tongue cells.

The invention provides a method of immunizing an animal, the method comprising the step of supralingually administering at least one antigen to the animal. In one embodiment, the at least one antigen is an attenuated virus, for example, an attenuated virus that infects tongue cells of the animal, or an attenuated virus that infects basal tongue cells of the animal. In one embodiment, the attenuated virus is canine parvovirus (CPV). In other embodiments, the attenuated virus is selected from foot-and-mouth virus, feline calicivirus, feline panleukopenia virus and feline parvovirus. In one embodiment of the invention, the attenuated virus is genetically engineered to present one or more antigens specific for one or more infectants in the animal.

In one embodiment of the method, the step of supralingually administering includes presenting the at least one antigen to the animal in a formulation which includes a solid matrix with the at least one antigen dispersed in the solid matrix, and with an abrasive substance dispersed in the solid matrix. In some embodiments, the step of supralingually administering includes a step of contacting a tongue of the animal with the solid matrix for a period of time selected from the group consisting of 1 minute, 2 minutes, 3 minutes, 4 minute and 5 minutes.

In another embodiment, the step of supralingually administering is performed a plurality of times over a period of weeks.

In yet another embodiment, the animal is a neonate, and in some cases, the step of supralingually administering is performed immediately after birth of the neonate and prior to a first provision of maternal colostrum to the neonate. In another embodiments, after the step of supralingually administering is performed, the neonate is not allowed to nurse for a period of time.

The invention also provides an immunogenic formulation for supralingual delivery to an animal, the immunogenic formulation comprising: a matrix; and at least one antigen dispersed in the matrix. In some embodiments, the matrix is a solid; and in some embodiments, an abrasive substance is dispersed in the matrix.

In one embodiment of the immunogenic formulation, the at least one antigen is an attenuated virus, for example, an attenuated virus that infects tongue cells of the animal, or an attenuated virus that infects basal tongue cells of the animal. In one embodiment, the attenuated virus is canine parvovirus (CPV). In other embodiments, the attenuated virus is selected from foot-and-mouth virus, feline calicivirus, feline panleukopenia virus and feline parvovirus. In one embodiment of the invention, the attenuated virus is genetically engineered to present one or more antigens specific for one or more infectants in the animal.

The invention further provides a supralingual applicator for administering an immunogenic composition to a puppy. The supralingual applicator comprises: a substrate at least including a matrix; and at least one antigen dispersed in the matrix. In some embodiments, the matrix is solid, and the matrix may comprise an abrasive substance dispersed therein. In some embodiments of the supralingual applicator, the at least one antigen is an attenuated virus, for example, a virus selected from CPV, foot-and-mouth virus, feline calicivirus, feline panleukopenia virus and feline parvovirus. In yet other embodiments, the supralingual applicator includes a protective plate positioned on one surface of the matrix.

### BRIEF DESCRIPTION OF THE DRAWINGS

FIG. 1. Schematic of a puppy tongue in longitudinal cross section.

FIG. 2A-G. Exemplary vaccine delivery applicators. A, supralingual applicator with holding means 10 attached to a ball of solid formulation 12 with abrasive particles integrated into formulation 13; B, cross-sectional view of delivery vehicle with holding means 10 with bristles (e.g. Luffa bristles) 14 protruding from support 15 which is coated with solid formulation 12; C, surface view of finger cot 16 coated with solid formulation 12 comprising abrasive particles 13; D, cross-sectional view of “pacifier” with support structure 17 and contiguous or attached support 18 coated with solid formulation 12 containing abrasive particles 13; E, side view of “pacifier” with support structure 17 with attached solid formulation 12 containing abrasive particles 13; F, cross sectional view of supralingual applicator with abrasive “teeth”; G, cross sectional view of supralingual applicator with abrasive “bumps” or “nubs”. In some embodiments, the antigen is also present, or is only present, in the bumps or nubs.

FIG. 3A-C. Schematic representations of various embodiments of a device to deliver the vaccine formulations of the invention. A, view of supralingual applicator's “top” surface, which does not contact the tongue, comprising holding means 10 and matrix 12; B, side view of supralingual applicator with holding means 10 and matrix 12; and C, view of supralingual applicator's “bottom” surface, which does contact the tongue, and of holding means 10 and matrix 12 and with grooves 19 in the form of a grid.

FIG. 4A-G. Schematic representations of various embodiments of a device to deliver the vaccine formulations of the invention. A, view of matrix showing surfaces; B, oval; C, rectangle, D, conical; E, substantially square; F, contoured supralingual applicator; and G, side view of supralingual applicator with matrix 20 and holding means 10.

FIG. 5A-D. Exemplary embodiments of the invention. A, layered matrix; B, matrix with a support or backing; C, supralingual applicator with protective plate over the matrix; D, exemplary lick block.

FIG. 6A-D. Fenestrated embodiments of a supralingual applicator. A, matrix with vertical channels; B, matrix with a variety of differently shaped channels; C and D, embodiments of matrices with channels opening on one surface [C] and going entirely through (D) the matrix. A handle 90 is connected via hinge 91.

FIGS. 7A and B. Exemplary protocols for vaccine administration. A, beginning at birth; B, beginning at 4 weeks. LP=supralingual applicator.

FIGS. 8A and B. A, schematic illustration of cat tongue showing filiform papillae with a caudally directed keratinzed spine arising from the caudal prominence; 100=lamina propia; 101=supporting rosta papilla; B, schematic illustration of a dog tongue showing filiform papillae with caudally directed apices; 200=apices; 201=lamina propria.

FIG. 9. Pie chart showing distribution of CPV in tongue vs intestines in the filed cases that were studied.

FIG. 10. Exemplary fluorescence in tongue of a dog infected with CPV showing the presence of the virus in basal cells. The circular raised area in the center is the taste bud.

### DETAILED DESCRIPTION OF THE INVENTION

The present invention provides compositions, devices and methods to apply or administer vaccines supralingually to mammals. According to an embodiment of the invention, antigens are delivered to the surface of the tongue (e.g. the dorsal surface) via a supralingual applicator which comprises a solid or semi-solid carrier of matrix containing one or more vaccine antigens of interest. The supralingual applicator may be disposable. The sucking and/or licking action of the tongue and contact with saliva dissolves the matrix, thereby releasing the antigens onto the tongue. The strong masticatory muscles of the tongue and mouth produce force and hence friction between the matrix and the tongue, helping delivery of the vaccine antigens. Since mammals have the innate ability to suckle naturally at a very early age, e.g. immediately after birth, the supralingual vaccine applicators of the invention may be used to safely and efficiently deliver vaccine antigens even to neonates. This strong suckling reflex also tends to clamp the matrix, holding it in place. Moreover, the solid and semisolid applicators pose no risk of aspiration as could happen with oral drops/liquids. The owner of the animal can deliver the vaccine to an animal in a facile manner, without needing to use a syringe or other vaccine delivery apparatus. Thus, supralingual delivery encourages compliance with vaccination protocols, especially perinatal vaccines that must be administered right after birth, e.g. in places where veterinary and medical services may be lacking.

In one embodiment, the method is used to vaccinate mammals against viruses that are capable of infecting one or more cells of the tongue (e.g. basal cells or other target tissue in and around the tongue), even though the tongue may not be a major site of infection, and infection of the tongue may thus not cause overt symptoms of infection. An exemplary virus of this type is CPV. Other non enveloped viruses that infect the tongue include viruses that cause foot-and mouth disease of cattle and feline calicivirus. In this embodiment, the vaccine preparation is administered in a manner that delivers an attenuated virus directly to the dorsum of the tongue of a vaccine recipient, e.g. at or near the basal cells or other target tissue of the tongue/mouth. In order to do so, a supralingual vaccine applicator is positioned within the mouth of the animal so that an attenuated-virus containing matrix portion of the applicator is in direct contact with the dorsal surface of the animal's tongue. This embodiment may be used even with newborns, and when the animal is a newborn, simultaneous contact of the matrix with the roof of the mouth elicits an innate suckling reflex, whereby the muscles of the tongue flex in a manner that dissolves the matrix and mechanically releases virus from the formulation. The shape of the applicators can vary, depending on the animal species, breed, size, mouth shape, etc. of the animal. Suitable applicators can be designed by using molds (e.g. plaster of paris, hard plastic, etc.) for animals of different types, sizes, ages, etc.

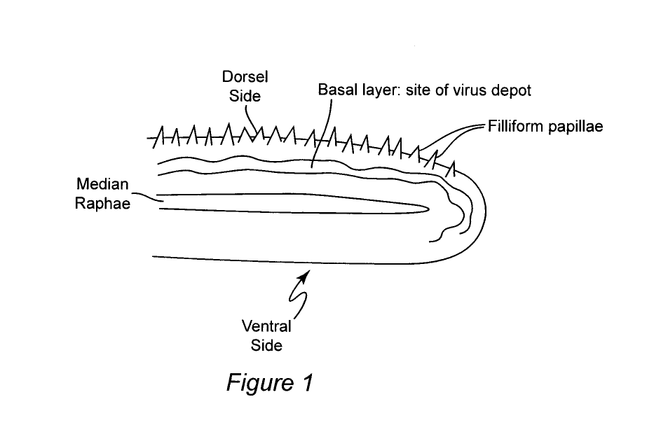

Without being bound by theory, it is believed that in this embodiment, the mechanism of supralingual vaccine delivery is as follows: Virus released from the applicator matrix via suckling, licking, etc. is transported by infecting the germinal layer of the tongue and is taken up by immune cells of the tongue. Lymphoid cells in the tongue initiate an immune response. In one embodiment, basal cells of the tongue are infected by the virus and an antigen depot (e.g. a reservoir of virus) is established. Over time (and from a very young age if the vaccine recipient is a newborn), the dividing basal cells gradually and persistently release virus and/or viral antigens into surrounding tissues. In supralingually vaccinated neonates, the virus infection is initially held in check and confined to basal cells due to neutralization of released viruses by circulating maternal antibodies. However, with time, maternal antibodies wane, and the juvenile animal's immune system gradually becomes active and takes over antibody production. Since the release of virus from the basal cell reservoir is ongoing throughout this transition, low levels of smoldering parvovirus infection are constantly present and, at an early stage, an active immune response to the virus is gradually produced by the maturing immune system of the animal. The invention thus provides a method of vaccinating newborns that bridges the gap between the time when maternal antibodies are active, and the time when the newborn's own immune system is competent leading to an active immune response that is protective, i.e. these time periods overlap. This view is consistent with the surprising discovery that, in CPV infected animals, viruses are located not only in the intestine and other predictable locations in the digestive tract (e.g. esophagus), but are also unexpectedly found in the basal layers of the tongue. This finding is described in detail in Example 1. FIG. 1 shows a schematic side view of the internal layers of the tongue, including basal cells.

### DEFINITIONS

It will be useful to define different types of vaccines.

Parenteral vaccines are traditionally the most common types of vaccines that are given subcutaneous or intra muscular or intra dermal with help of needle and syringe. The vaccine preparation is normally in a solution or liquid form. These vaccines have been used for more than 200 years (introduced by Edward Jenner in 1796) and some of the vaccines have been effective. One of the most successful vaccines is the small pox vaccine that is delivered by spilt needle in the skin. Because the poxvirus is a skin tropic virus, the correct type of immune response provides solid lifelong immunity. Most vaccines against mucosal infections given by parenteral route may not elicit the correct type of immune response. Accordingly, sometimes these vaccines are not effective. Moreover, parenteral vaccines can cause adverse reactions in many cats and dogs due, for example, to the presence of adjuvants (e.g. injection site sarcomas in cats).
Oral Vaccines Some orally delivered vaccines (such as polio drops) have been effective. One of the limitations of the oral delivered vaccines is the degradation of the vaccine antigen by the low pH of gastric acids.
Intranasal vaccine: These are vaccines that are used as e.g. drops in the nose or as an aerosol, usually for respiratory pathogens. While sometimes efficacious, nasal vaccines can also lead to adverse reactions due to transport of antigens to the brain.
Sublingual vaccine: Another method of vaccination is sublingual vaccination. It can be implemented using a liquid or a solid tablet given below the tongue. One advantage of sublingual vaccine is that it can provide tolerance to some allergens because the reduced volume of vaccine is deposited on the underside of the tongue. However, the sublingual route is not convenient because the tongue has to be lifted to deposit the vaccine.
Supralingual vaccine: The supralingual vaccines of the invention overcome the limitations of the previously described vaccination routes and allow easier application of vaccines. One aim is to allow enough contact time for administration of a sufficient dose of antigen to immunize, e.g. a new born mammal. Supralingually delivered formulations make enough contact with the tongue, and for a sufficiently long period of time, to allow antigen delivery. Further, the contact is intimate and with pressure by the powerful masticatory force of the jaw muscles, which may encourage faster dispersal of the antigen and thus facilitate antigen delivery. This is accomplished and/or facilitated by the design features of the supralingual vaccine delivery device, i.e. the supralingual applicator. Supralingual approaches take advantage of the natural suckling reflex and the natural mechanical brush like structures on, for example, the dorsum (upper surface) of the tongue. A supralingual vaccine is delivered in situ in reduced volume as, e.g. a solid, semisolid or gel, such as an in situ formed gel. Moreover, the supralingual cavity is larger than the sublingual space and this allows applicators to be shaped for as to promote even application, e.g. a rounded shape which is easy to roll on the tongue. Another advantage of the supra lingual approach is the lower temperature of the mouth (by about 1-2° C.) which allows higher stability of fragile viruses. Some viruses, such as parvovirus, are exceptionally stable and thus are readily incorporates into the applicator matrix. The proteolytic but non-denaturing action of saliva also facilitates the proper integrity of the vaccine. Further, administration in this manner results in a reduced risk of delivery of the virus to the brain of the animal, compared, for example, to systemic or nasal vaccine administration. Further, the lack of adjuvants reduces the cost or production and the occurrence of adverse reactions. Moreover, very few, if any, safe food or feed grade adjuvants are currently available. Most parenteral adjuvants elicit intense inflammation and thus are uncomfortable and unsuitable for application on the tongue.

Supralingual vaccination solves the problem of variation of maternal antibody titers, e.g. against CPV. In fact, in cases where maternal antibody titers are low, active immunity may develop earlier in animals that are vaccinated supralingually. Delivery of the vaccine, for example, to the dorsal surface of the tongue can be accomplished by any of several methods. For example, the vaccine may be delivered using in a hand-held “supralingual applicator”. The applicator typically includes a solid or semisolid matrix/carrier that contains the antigens of interest, the matrix being in a form or shape that is suitable for contact with the dorsal aspect of the tongue of a mammal. In one embodiment, the matrix is formed so as to be suitable for placement within the mouth in a manner that positions a surface of the matrix on the dorsal surface of the tongue, i.e. at least one surface of the matrix makes direct contact (or is directly contactable) with the dorsal surface of the tongue, e.g. by suckling, licking, or other flexing motion of the tongue muscles. In this embodiment, a handle or other holding means may be attached to the matrix in order to facilitate or control administration and prevent swallowing of the matrix. In other embodiments, the matrix is fashioned primarily for licking and need not necessarily be of a size or shape that can be accommodated by a mammalian mouth. In some embodiments, the design and size of the applicator is chosen to allow or facilitate contact, and in some cases, maximal contact, with the dorsal surface of the tongue (e.g. at least about 10, 20, 30, 40, 50, 60, 70, 80, 90, or 95%, or even more of the dorsal tongue surface). Most adult animals have a natural, inherent ability to extend the tongue in order to taste and/or lick substances. The hardness of the matrix described herein generally prevents biting but promotes or encourages licking of the applicators. Packing of the matrix and/or applicator in vacuum packs helps to maintain crisp taste and prevent spoilage. These and other embodiments are discussed in detail below.

In some embodiments, the supralingual applicator comprises a comestible or edible (e.g. food or feed grade) matrix/carrier in which one or more antigens of interest are present. Upon contact of the matrix with saliva in/on the dorsum of an animal's tongue, the matrix dissolves or disintegrates, thereby releasing antigens to the dorsal surface of the tongue. Exemplary matrices of this type include but are not limited to: a solid/semi-solid “lollipop” style device (that may be scored to allow size reduction by breaking into smaller sections) that is placed on the tongue and allowed to dissolve; a liquid which becomes solidified or gelled when applied to the tongue (e.g. see B. Madan, Bajaj, et al. Indian Journal of Pharmaceutical Sciences 2009. 71:242-251 for a description of in situ forming polymeric drug delivery systems; a sheet of dissolvable material; a frozen “popsicle” type matrix; etc. Such matrices are generally entirely consumable, e.g. after sufficient tongue contact has been achieved. In this embodiment, the applicators are for use with one individual animal, thus preventing transmission of mouth infections to other animals. The size, shape and dimensions of the applicators can be tailored, depending on e.g. the breed, size of the mouth, body weight, chewing strength, and preferences of the animal receiving the vaccine.

In other embodiments, the supralingual applicator may include a substantially durable solid or semi-solid substrate or support that is not consumable/edible/comestible and does not dissolve upon contact with saliva, but which acts as a vehicle for delivering antigen to the tongue. In this embodiment, antigen is released from the durable vehicle by the suckling or licking action of the tongue. The substrate may be a handle or holding means, one portion (e.g. an end) of which is embedded in the matrix, and another portion (e.g. the other end) of which protrudes from the matrix and can be grasped by a user of the applicator to facilitate administration of the vaccine to an animal. The antigens may be present in a solid or semi-solid matrix that is attached to or positioned on or within the support, or a matrix per se may be absent, the vaccine preparation which contains antigens being coated (e.g. dried) directly onto the support or infused or impregnated into the support. Examples of this embodiment include but are not limited to: the coating of a finger cot or sheath which is offered to an animal; bristles (e.g. Luffa bristles) coated or impregnated with vaccine formulation; a sponge or sponge-like material coated or impregnated with vaccine formulation; puppy pacifier coated or impregnated with vaccine formulation; a non-dissolvable sheet of material which is coated or impregnated with matrix or vaccine formulation, etc. The substrates may be disposable or reusable. Reusable substrates may be repeatedly coated with matrix, administered, cleaned, re-coated, administered, cleaned, etc.

In yet other embodiments, the vaccine formulation is delivered as a liquid or semi-solid formulation (e.g. a thick liquid, syrup or paste) that is painted or rolled onto the tongue. This is unlike drops which will be immediately ingested and which do not allow sufficient contact time between the antigen(s) and the dorsal surface of the tongue.

Administration of antigens according to the invention occurs via direct contact with the surface of the tongue (e.g. the dorsal surface), and over a period of time. Generally, contact time is greater than 1 minute, may be 5 minutes or more, e.g. usually about 10 minutes, and may be as great as 15-20 minutes. Contact time can be adjusted by adjusting the hardness of the matrix, and/or by limiting the time during which the matrix comes into contact with the tongue (withdrawing the applicator), etc.

In some embodiments, the solid or semi-solid formulations contain abrasive substances or particles such as “grit” or “crystals” which promote light scoring or abrasion of the tongue. Examples of such substances include but are not limited to: crystallized sugars, salts, minerals, etc; finely divided particulate kibble or other comestible substance that is hardened, e.g. that is dehydrated (e.g. baked or otherwise heated) until of a very hard consistency; etc. Care is taken to utilize only substances that cannot harm the animal (e.g. food or feed grade components for the targeted species). In other embodiments, when the supralingual applicator includes a durable substrate or support, the support itself may include protrusions that gently abrade the animal's tongue as it sucks or licks, e.g. short (e.g. 1-5 mm) bristles or other hard surfaces that gently rub against the tongue surface. When the antigen that is delivered is a virus, the purpose of slightly wounding, scoring or otherwise irritating or abrading the tongue (without, however, being painful) is to more efficiently deliver the virus, e.g. to the basal cells of the tongue. The basal cells are generally located approximately 10 cell layers deep, and the solid vaccine formulation is generally designed to, in response to the sucking action of the animal, create pathways to deliver the virus in the vicinity (e.g. within 2-5 cell layers) of the basal cell layer.

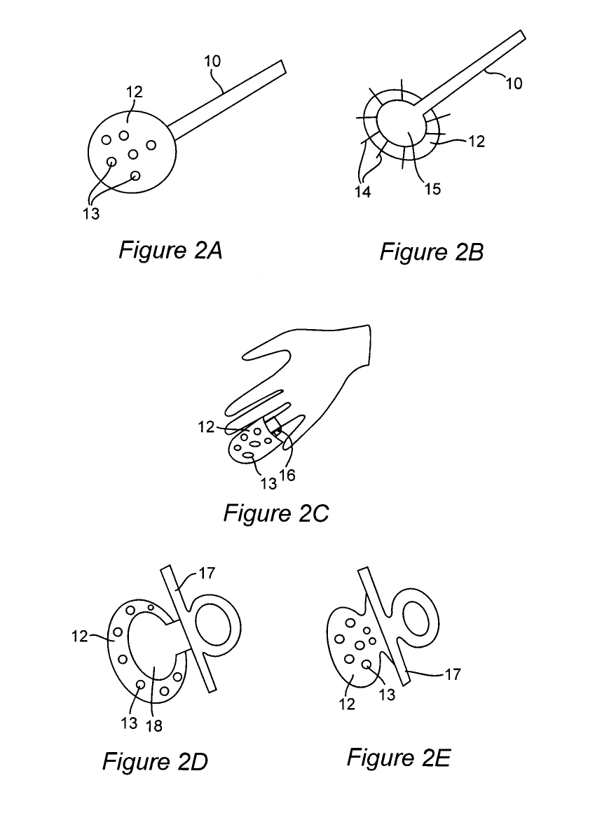

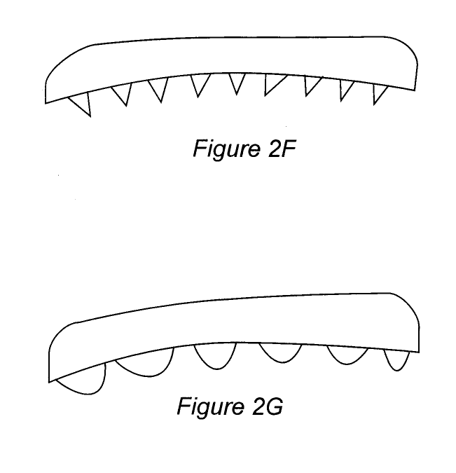

Several exemplary vaccine delivery devices are shown schematically in FIG. 2, where FIG. 2A depicts a “supralingual applicator” with holding means 10 attached to a ball of solid formulation 12 with abrasive particles 13; FIG. 2B shows a cross-sectional view of a delivery vehicle with holding means 10 and bristles 14 protruding from support 15 which is coated with solid formulation 12. The holding means is generally a handle, and may be detachable and/or may vibrate (oscillate, pulsate, e.g. 500-30,000 strokes per minute) e.g. via a battery powered mechanism in order to enhance dissolution of the matrix onto the tongue, and in some embodiments, may deliver ultrasonic waves. FIG. 2C is a surface view of finger cot 16 coated with solid formulation 12 comprising abrasive particles 13. FIG. 2D is a cross-sectional view of a “pacifier” with support structure/holding means 17 and contiguous or attached support 18 coated with solid formulation 12, which contains abrasive particles 13. FIG. 2E is a side view of a “pacifier” with support structure/holding means 17 with attached solid formulation 12 containing abrasive particles 13. Other embodiments are illustrated in FIGS. 2F-G, and include the incorporation of “teeth” or “bumps” or “nubs” on the surface of the supralingual applicator, either formed from the solid matrix material itself, or provided on a support or substrate on which the matrix is affixed, and extending through the matrix so as to contact the tongue during licking or sucking of the device. In some embodiments, the antigen is present in the abrasive protrusions (e.g. in the bumps, nubs, bristles, etc.) and may be present only in the abrasive protrusions.

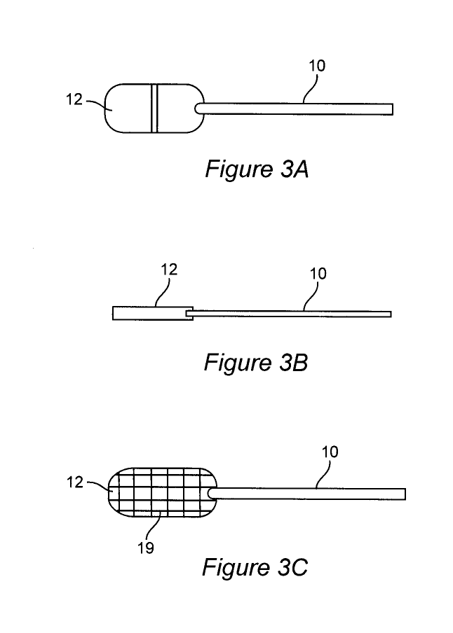

In other embodiments, one or more surfaces of the supralingual applicator are grooved or striated, e.g. with indentations or open channels which run along the surface of the supralingual applicator, to allow some “catching” of the papillae of the tongue. The edges of the grooves or channels may be irregular or rough to provide mild, abrasive action. This embodiment is illustrated in FIG. 3A-C, where the grooves are depicted in a grid pattern on the bottom surface of the supralingual applicator (the surface which contacts the tongue, FIG. 3C).

In some embodiments, the delivery device is a chew toy made, e.g. from natural rubber for strength and durability, that is impregnated with vaccine. In certain embodiments, the chew toy is flavored to insure (encourage) chewing or mouthing of the toy. In some embodiments, raised nubs on the toy or pacifier help to slightly abrade the tongue.

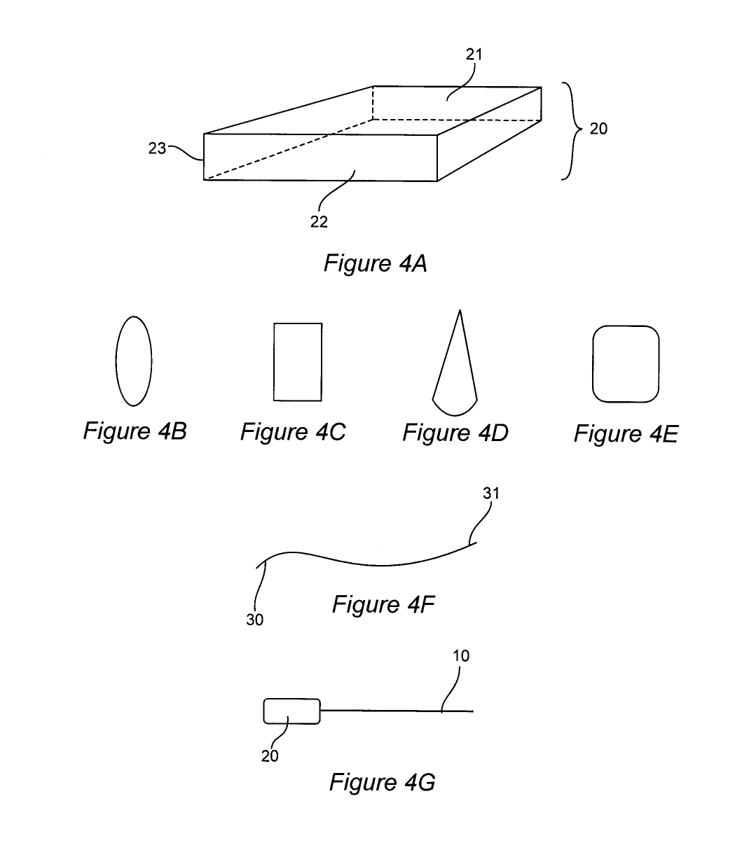

Further exemplary embodiments of a vaccine supralingual applicator are depicted in FIG. 4A-G. For example, the supralingual applicator may be of any suitable size and shape, so long as it fits comfortably into the mouth of the animal and delivers the viruses to the dorsal surface of the tongue. For example, with reference to FIG. 4A, matrix 20 may have a top surface 22, a bottom surface 22 (which is not visible in the Figure, and may be considered to be coincident with the plane of the paper) and sides 23. The shape of the top (upper) and bottom (lower) surfaces of a supralingual applicator may be e.g. substantially: oval (FIG. 4B), circular; a regular polygon (e.g. substantially square as in FIG. 4E, or rectangular as in FIG. 4C, etc.) or an irregular polygon, or conical on shape (FIG. 4D), etc., when viewed from above (or below). Any suitable shape may be employed. In some embodiments, delivery vehicles are designed so as to accommodate the size and anatomy of an animal's mouth. In some embodiments, delivery vehicles are designed for intra-oral delivery, i.e. so as to accommodate the size and anatomy of an animal's mouth. Generally the longest dimension (e.g. of a top or bottom surface) will be in the range of from about 1 to about 5 cm, and usually in the range of from about 1.5 to about 3 cm, e.g. about 1, 2, 3, 4, 5 or more cm (e.g. 10-20 cm or more), depending on the size of the mouth and tongue of the vaccine recipient. In adult dogs, the tongue may be 20 cm long. A larger and longer applicator can be used for adult animals. With respect to thickness, the supralingual applicator is generally in the range of from about 0.25 to about 1.5 cm, and usually in the range of from about 0.5 to about 1 cm, i.e. from about 0.3, 0.4, 0.5, 0.6, 0.7, 0.8, 0.9, or 1 cm, in the thickest portion of the supralingual applicator. The supralingual applicator may have a substantially consistent thickness throughout, i.e. the top and bottom sides of the supralingual applicator may be substantially parallel, or one or both of the top and bottom side may be curved. For example, the overall shape of the supralingual applicator may approximate a sphere, an ovoid, etc, in which case no “sides” per se are present. Alternatively, the supralingual applicator may be shaped or configured so as to approximate the internal shape of an animals mouth, e.g. as depicted in FIG. 4 F, which shows a side view of a supralingual applicator with curved front section 30 and curved back section 31. In some embodiments, the supralingual applicator comprises a holding means (10 in FIG. 4G) such as a handle to facilitate manufacture, handling and administration of the device. A handle will generally be straight and in the range of from about 5 to about 10 centimeters in length, or sometimes shorter, and can be rigid or flexible. However, various other designs are possible, e.g. in which the holding means is bent or curved, or wraps around the finger or hand of the person who is administering the vaccine, etc. The disposable embodiment of the applicator prevents cross-contamination between puppies. Dog owners can order the correct size applicator prior to birth of the puppies, e.g. based on experience with the breed and the size of a pregnant females mammary gland nipples, so that the vaccine applicators are available for use shortly after birth.

The supralingual vaccine applicators of the invention may comprise a solid or semisolid matrix which contains antigens dispersed or distributed throughout, the matrix being dissolvable upon contact with saliva. The hardness of the matrix can be controlled by the temperature that is used during manufacture. Those of skill in the art will recognize that any of several suitable types of matrices can be employed. For example, a “lollipop” or hard candy-style formulation may be used.

Like most hard candy recipes, vaccination applicators can be produced by cooking sugars. An exemplary combination is sugar (2 cups), light corn syrup (about ⅔ cups), ¼ cups water and salt (⅓ teaspoon). Coloring and different favors such as meat or peanut can also be added. First the ingredients are boiled to 300° C. without the lid cover on the boiler. Care should be taken because this mixture has lot of latent heat and can burn the skin. Let the mixture cool. After the temperature falls down to about 125° C. then antigens can be mixed in uniformly for even dispersal. The mixture is poured into molds to harden. The hardness of the candy can be monitored by the well-known cold water test, and more correctly (accurately) with a candy thermometer to achieve the desired level of hardness.

Depending on the desired level of hardness or brittleness, the boiling time of the candy mixture and the temperature achieved can be adjusted and the stage of hardness monitored, e.g. using a “cold water test” as is known in the art and described in Table 1. However, it is advisable to also measure the temperature using a thermometer. Exemplary cold water test stages and the corresponding temperatures are as follows:

TABLE 1
The Cold Water Test

| Stage | Temperature | Characteristics |
|---|---|---|
| Soft Ball Stage | 234-240° F.; | Mixture forms a soft ball that |
| | 110-115° C. | flattens when removed from water |
| Firm Ball Stage | 242-248° F.; | Mixture forms a firm ball that holds |
| | 115-120° C. | its shape until pressed |
| Hard Ball Stage | 250-268° F.; | Mixture forms a ball that holds |
| | 120-130° C. | its shape but is pliable. |
| Soft Crack Stage | 270-290° F.; | Mixture separates into hard but |
| | 130-145° C. | not brittle threads. |
| Hard Crack Stage | 300-310° F.; | Mixture separates into hard, |
| | 150-155° C. | brittle threads. |
| Caramel Stage | 320-350° F.; | Do not use cold water test; |
| | 160-175° C. | mixture coats metal spoon and |
| | | forms light caramelized mass |
| | | when poured on a plate |

Altitude above sea level affects the boiling temperature of the liquid and can be empirically determined using a thermometer.

In some embodiments, the matrices which contain the antigens are solid, i.e. they have a definite shape and volume, are firm and do not spread and are not readily malleable or surface deformable (although they may be flexible). Rather, they retain their shape at temperatures such as those which are encountered in the body of an animal (e.g. dog=about 37-38.6° C.), e.g. in an animals mouth (1-2° C. lower), and at room temperature (e.g. about 25° C.). Those of skill in the art will recognize that the degree of hardness of the matrix may vary somewhat, depending on the design of the supralingual applicator. Methods for manufacturing suitable matrices, and for analyzing their physical properties, may be found, for example, in U.S. Pat. No. 6,455,096, the complete contents of which is hereby incorporated by reference.

In one embodiment, the vaccine can be blended with peanut butter and loaded or soaked in a rubber unit that is commercially available, such as a Kong or rope toys. These toys are most suitable for booster shots in puppies. These toys can be washed in a dishwasher. Moreover, the “lollipop-style” devices are more suitable for newborn dogs.

Other substances may also be included in the formulations, e.g. substances that are beneficial such as vitamins, neutraceuticals, iron supplements, other medicaments, deworming agents such as Ivermectin, etc. Also, various flavorings or other palatable substances that are likely to appeal to a puppy may be included, e.g. meat, fish, milk, vegetable, or other flavorings. In addition, adjuvants, especially mucosal adjuvants such as bacterial toxins (e.g. cholera toxin), may be included. In some cases, rinsing with a mouth wash containing iron salts is used after vaccine administration to stop cell binding and infection by and/or compete with cell binding and infection by left over or residual virus such as CPV (i.e. with virus that has not yet entered a cell).

Other suitable ingredients for inclusion in the vaccine formulation include but are not limited to: alcohols, various release controlling additives (e.g. hydroxy propyl methyl cellulose, hydroxy ethyl cellulose, hydroxy propyl cellulose and polyethylene glycol and like polymers). The formulation may contain: known pharmaceutically acceptable additives, flavoring agents, surfactants (e.g. non-ionic surfactants) and adjuvants; various stabilizers; antioxidants; fillers; buffering agents; glycerol; sugar or other natural and artificial sweeteners. Salts are included in the compositions in particular, since they tend to promote salivation and thus facilitate release of antigens from the supralingual applicator. Buffering agents to maintain a suitable pH for oral administration and/or to be suitable for a virus contained in the matrix (e.g. about pH 7.2 for canine parvovirus and pH 6.5 for feline panleukopaenia virus). Generally, the active ingredient(s) (e.g. one or more antigens, or other medically beneficial substances) is/are present in the formulation is an amount ranging from 1-99%. When the antigen is a virus, the amount of virus is generally in the range of from about 10⁴ to about 10¹⁰ virus particles per supralingual applicator, and usually in the range of from about 10⁵ to about 10⁷ virus particles per supralingual applicator. This much virus is generally present in about 100 ul-200 ul of cell culture supernatant. The level of attenuation of live viruses (e.g. CPV) is controlled by the passage number. The range of passages need for modifying live viruses can be 25-100, with 25 being an exemplary passage number. Most CPV viruses in parenteral injections are about 75 passages.

In some embodiments, immune enhancers are also present in the formulations. For example, whey proteins, Spirulina platensis, probiotics such as lactic acid bacteria (e.g. Lactobacillus rhanmous, L. acidophilus), bifidobacteria (e.g. Bifidobacterium lactis), or various streptococcus species.

As will be evident from the descriptions presented above, the delivery devices of the invention may be entirely consumed by suckling and licking of the soluble matrix by an animal during administration of a vaccine, e.g. the entire supralingual applicator may dissolve. However, this need not be the case. The vaccine-containing formulation may be coated onto or impregnated into a substrate or support and licked or sucked off, leaving the substrate behind. In such embodiments, the substrate may, for example, be fashioned from any suitable material that is able to retain a coating of the vaccine formulation, or to incorporate vaccine formulation within the substrate (e.g. within holes or channels in the substrate, or generally soaked into the substrate). Examples of such materials include but are not limited to various soft flexible fibers (both natural and synthetic); cloth; leather or rawhide strips; “chewy” materials such as those from which pacifiers and chew toys (KONG company, Golden, Colo.) are made; etc. Further, liquid vaccines can be mixed with peanut butter and frozen to allow or encourage licking and discourage quick swallowing of the vaccine.

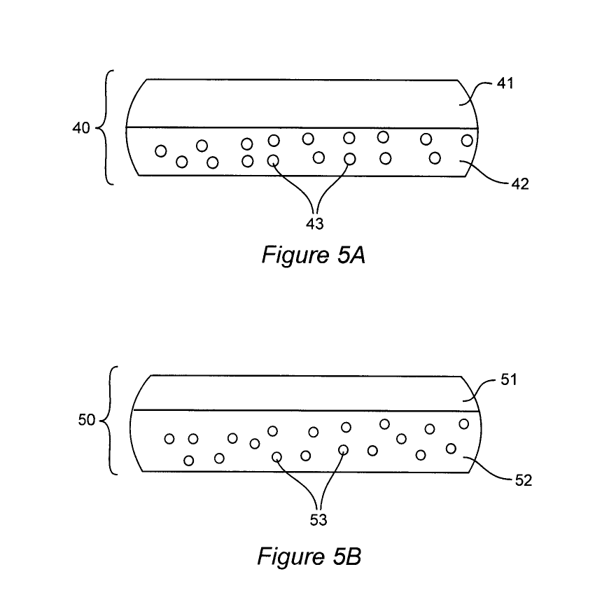

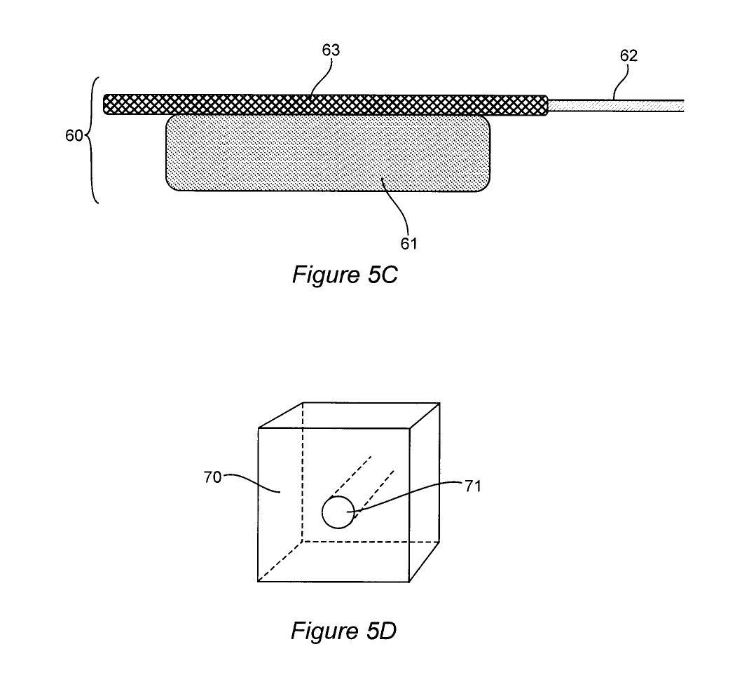

In addition, in some embodiments of the invention, the vaccine formulation is present on only one side of the supralingual applicator, e.g. is coated on or impregnated into only one side of a substrate, or is layered so that only one side of the supralingual applicator contains the vaccine, and the other side does not. In this embodiment, the side of the device which includes the vaccine components is placed on the dorsal surface of the animals tongue and the opposing side is poised next to the roof of the animals mouth. This embodiment is generally used for very young animals who are relatively quiescent, and for whom it is possible to stably manipulate the position of the device within the mouth. This embodiment advantageously delivers the vaccine preparation directly to the surface of the tongue, with very little being delivered to the other parts of the animals mouth. The surface of the device that does not include the vaccine components may be formed from a solid or semi-solid comestible substance that is the same as that of the vaccine-containing surface, but minus (without) the vaccine, e.g. the device comprises at least two layers, only one of which includes vaccine. This embodiment is illustrated in FIG. 5A-C, where in FIG. 5A supralingual applicator 40 is shown as including inert (non-vaccine) upper layer 41 and a lower layer 42 which comprises vaccine components 43. Alternatively, in this embodiment, the non-vaccine containing surface may comprise an inert, non-comestible substance that serves as a support or backing for the delivery of the vaccine formulation, e.g. a strip of paper or cardboard, cloth, synthetic polymer, etc. This embodiment is illustrated in FIG. 5B, where supralingual applicator 50 is shown as comprised of lower layer 52 comprising vaccine components 53, coated or attached to substrate or backing 51. This version is also depicted in FIG. 5C, which schematically depicts a side view of a supralingual applicator 60 which comprises vaccine-containing matrix 61 (which comes into contact with the tongue), holding means 62, and protective plate 63, which extends or protrudes out over a surface of matrix 61, does not come into contact with the tongue, but usually with the roof of the mouth, and prevents the animal from biting or chewing the matrix. Combinations of these are also contemplated, e.g. a layered supralingual applicator construction that also includes a substrate or backing.

In some embodiments, the supralingual applicator of the invention is rigid in construction, i.e. the form or structure of the supralingual applicator is generally retained until it is dissolved by contact with saliva. In other embodiments, the supralingual applicator is not rigid but is flexible. In the latter embodiment, the formulation may be, for example, a gel or other substance that contains the vaccine and is dissolved by contact with saliva; or a flexible gel-like substance (e.g. various edible plant gums or thick pastes) that is impregnated with the vaccine (with or without also being coated on a support, depending on the degree of flexibility), and which does not dissolve upon contact with saliva. Such formulation may or may not be positioned on a support for delivery of the vaccine to the animal.

In an exemplary embodiment, a solid formulation comprising attenuated CPV virus and mildly abrasive sugar crystals is prepared and administered to a puppy less than 8 weeks of age by suckling. The puppy may be a neonate. Mild abrasion of the puppy tongue by sucking action results in delivery of the attenuated virus to the basal layers of the puppy's tongue. The attenuated CPV infects the basal cells of the tongue and establishes a low level persistent infection. Viruses escaping from the basal layer elicit an immune response to CPV, resulting in the production of anti-CPV antibody production by the puppy.

In some embodiments, the solid or semisolid matrix is imprinted or embossed with an indication of the type of antigen that is contained therein, e.g. if the antigen is a virus, D may be used for distemper, P, for parvovirus, K-9 for canine, etc.

In one embodiment, the invention provides large lick blocks (e.g. salt licks and/or urea blocks) to promote domestic livestock (e.g. ruminant livestock) and wildlife vaccination using the supralingual approach. Salt licks are frequently used to provide the minerals such as phosphorus, sodium, calcium and magnesium to ruminants. Stable vaccine compositions may be incorporated into the lick blocks during manufacture, or may be painted or soaked into the lick blocks. For example, salt and mineral blocks are generally porous and can soak up liquid vaccine if immersed therein for e.g. about 30 minutes. They can then be offered to animals. This embodiment may be useful for either a juvenile or adult animal, and can be used e.g. to vaccinate domestic livestock (e.g. cows, goats, sheep, llamas, horses, etc.), or animals in protected areas or rescue facilities (e.g. zoos, animal parks, animal shelters, etc.), or wildlife (e.g. foxes, raccoons, deer, etc). A schematic representation of lick block 70 is provided in FIG. 5D. In this representation, optional hole or channel 71 is provided for hanging of the block. In one embodiment, lick blocks with antigens directed against foot-and-mouth disease (e.g. killed viral antigens) are provided.

In exemplary embodiments, commercial salt licks are purchased and impregnated with a solution which contains one or more of the vaccine antigens described herein, especially formulated for supralingual administration. Alternatively, a salt lick can be formulated to include the one or more antigens together with other constituents, e.g. powdered bone (bone meal), shells, ashes, etc., plus various salts and minerals (e.g. rock salt), and one or more binders such as clay, cement, etc, formulated with sufficient liquid (e.g. water) to make a paste. For example, in one embodiment, a salt lick is made by combining two parts rock salt, four parts bone powder and one part of termite clay, enough water to create a paste, and a suitable dosage of antigens. In another embodiment, a salt lick (10 kg in weight), comprises 6.3 kg of salt, 1.5 kg of bone meal, 0.6 kg of ashes and 1.6 kg of cement, liquid to form a moldable paste, and a suitable dose of antigens as described herein. Yet another embodiment comprises: salt or mineral salt 82%; bone meal 4%; lime (crushed shells or agricultural lime) 2%; cement, good clay or a mixture of the two 12%, plus antigens. Other suitable substances such as molasses can be added to sweeten and help bind the mixture. Molasses should be added before the water since it will also provide some moisture. The paste is molded in a suitable container (e.g. an aerated wooden or metal box, bowl, can, etc.). Once dried, the salt lick is ready for use. The block is dipped in a vaccine solution and allowed to soak to impregnate the lick block with vaccine, which permeates the block by capillary action.

### Antigens

The supralingual applicators of the invention are designed to carry out the supralingual delivery of antigens. Examples of antigens that may be delivered by the applicators of the invention include but are not limited to: antigenic proteins, polypeptides, and peptides, e.g. those which are known to encompass or include epitopes or antigenic regions of disease-causing agents such as viruses, bacteria (i.e. bacterial antigens) various parasites, etc. In one embodiment, the disease-causing agents are those which cause dental caries, and the formulations of the invention include bacterial antigens are used as vaccines against dental caries. Suitable doses of antigen may be provided, e.g. by repeated and/or timed intervals of administration.

In one embodiment of the invention, the antigens include one or more viruses (preferably attenuated viruses) to which it is desired to induce or elicit an immune response in the vaccine recipient. “Viruses” includes whole viruses (live, live attenuated, killed, etc.), modified viruses (e.g. capsids containing nucleic acids), various infectious particles, viral subunits, virus-like particles, and other viral forms. In some embodiments, the viruses are genetically engineered to contain and express nucleic acids encoding one or more antigens specific for one or more infectants (e.g. disease causing agents). In some embodiments, the viruses have the ability to infect or to be taken up by basal cells. By “virus” or “attenuated virus” we mean a virus as is understood in the art, e.g. a virion or virus particle that generally includes nucleic acid material (and sometimes proteins), packaged within a protein coat, and which may or may not also contain a lipid envelope. An advantage of using live virus vaccines is that detectable immunity against the specific viral pathogenic agent usually develops quickly, e.g. in about 5 days to 2 weeks. Generally, viruses contained in the compositions are attenuated, i.e. they have been modified so that they do not cause disease in a recipient. However, such viruses retain components and/or structures which elicit an immune response to the virus, e.g. they retain epitopes, antigenic determinants, etc. to which the recipient mounts an immune response. Preferably, the immune response is a protective immune response, i.e. the response generates sufficient protection to prevent, or at least to lessen the degree of, symptoms of disease which would otherwise occur when the vaccine recipient is exposed to or challenged with a wild-type, disease causing virus which comprises the same or similar antigens. Those of skill in the art are familiar with methods for attenuating viruses, which include but are not limited to methods described in U.S. patent application Ser. Nos. 12/138,085 (filed Jun. 12, 2008 and published as US 2009-0010955) and 12/211,174 (filed Sep. 16, 2008 and published as US 2009-0098152), and in U.S. Pat. No. 7,744,902, the complete contents of all of which are hereby incorporated by reference. For example, one such procedure involves subjecting a virus to serial passage in cell culture, e.g. at progressively lower, attenuating temperatures. Alternatively, specific mutations can be introduced by subjecting a parent virus to chemical mutagenesis, (e.g. replication of the virus in the presence of a mutagen such as 5-fluorouridine, 5-fluorouracil, nitrosoguanidine, etc.). Combinations of these techniques may also be used. Preferably, the attenuated virus retains the ability to infect basal cells of the tongue, although in some embodiments, killed viruses may also be delivered. Killed chemically inactivated viruses bind to cells and are internalized but do not replicate in cells.

In one embodiment of the invention, the virus that is delivered is adapted to replicate at temperatures that are slightly lower (e.g. by 1-2° C.; e.g., at temperatures of about 35-36° C.) than core body temperature by in vitro cultivation and adaptation at these lower temperatures. The adaptation to slightly lower temperature also provides higher safety against potential ingestion, since the attenuated virus will not survive within the gastrointestinal tract, which has a temperature of 37-38° C. Thus, the virus is safe even when delivered in high doses (as high as 10⁷ to 10⁸ viruses per puppy. Most parenteral vaccines deliver 10⁴-10⁵ per puppy. In the case of parvovirus infection of puppies, the viral DNA could be present throughout the body of a vaccinated puppy but productive replication would occur only in the tongue and intestines. Thus, stability of viruses in the slightly lower temperature of the mouth facilitates supra lingual vaccine antigens. In addition, salivary pH, which is not extreme in most animal species, may serve to stabilize the live virus.

In some embodiments, the virus that is administered in the composition is a parvovirus, for example, an attenuated canine parvovirus, and the recipient is a canine. However, vaccination against other types of viruses and etiological agents is also encompassed by the invention. Exemplary viruses that can be delivered via the compositions of the invention include:

1) Non-enveloped viruses, which include but are not limited to: for puppies, canine parvovirus and canine adenovirus; for cats and kittens, parvovirus, calicivirus and feline panleukopenia virus; for calves, rota virus; for foals, rotavirus; for carnivorous wild life, parvovirus; apthovirus which causes foot-and-mouth disease in cattle and other cloven-foot animals (also known as hoof-and-mouth disease, the #1 serious viral disease of cattle in many parts of the world); etc.
2) Enveloped viruses, which include: for puppies, canine distemper virus and canine influenza (H3N8); for kittens, feline herpes virus; for calves, corona virus; for cattle, viruses which cause bovine viral diarrhea and bovine herpes virus; for foals, herpesvirus; for carnivorous wild life, distemper virus and rabies virus etc,

For delivery of non-enveloped viruses (parvovirus, rotavirus) the virus may be generally incorporated into (e.g. mixed or distributed within) the supralingual applicator matrix (e.g. a solid or semisolid formulation as described herein), usually during formulation of the matrix. As such, the virus is a component of the matrix, and is generally distributed relatively uniformly throughout the matrix. On the other hand, layered and/or other various non-uniform distribution patterns of the viruses are also included in the present invention. In such embodiments, if the matrix is a hardened material similar to classical supralingual applicators, and is made by boiling water and a saccharide such as conventional sugar (sucrose), maple syrup, etc., then the viruses that are added to the supralingual applicator mix are generally stable in a temperature range of from at least about 100-150° C., or from at least about 110-140° C., or from about 120-130° C. This stability and viability should persist for at least a short period of time while the virus is added to a cooling mixture, but before the mixture hardens. Other formulations may also be possible, e.g. in which the matrix with virus is baked or dried with or without heat, or with only minimal heat, thereby obviating the necessity for heat stability. In some embodiments, mixing the virus with a peanut product (e.g. peanut butter) is also recommended in case the virus is very labile.

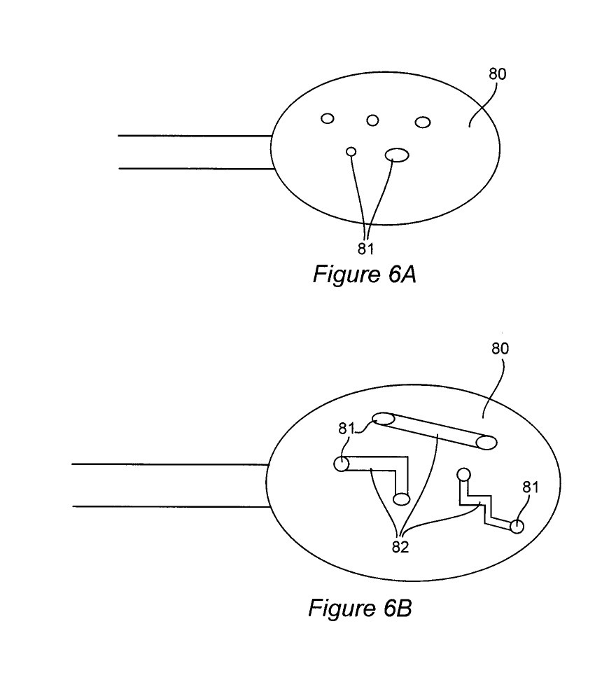

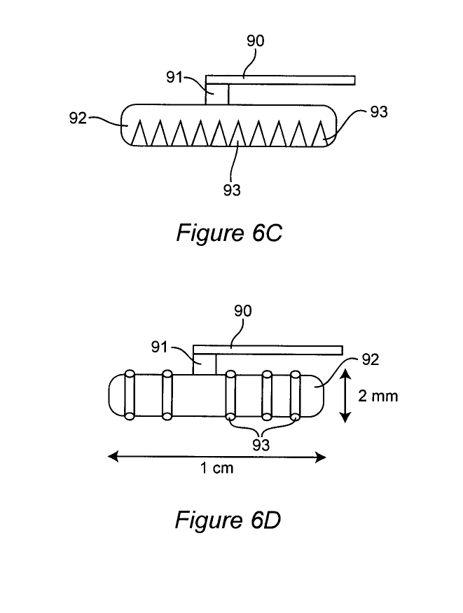

For delivery of enveloped viruses, a delivery vehicle (device) with capillary holes as illustrated in FIG. 6A-D is contemplated (although this type of device may also be used for non-enveloped viruses, or for other antigens as well). With reference to FIG. 6A, in this embodiment, the solid or semi-solid matrix 81 has distributed therein a series or network of relatively uniform capillary holes 81 (openings), channels or chambers which permeate the matrix, i.e. the matrix is fenestrated. The volume of these capillary holes or channels is substantially uniform and generally ranges from about 1 ul to about 10 ul, and the number of holes per square centimeter of matrix is in the range of from about 10 to about 500 or more. The channels may form an interconnected network, or be tangential (side-by-side), and may be of any relatively or substantially uniform shape, e.g. substantially straight, curved, angular, etc. They may have any suitable diameter, so long as the vaccine composition can enter and fill the channels, and be sequestered therein until administration of the vaccine. At least one portion of the holes/channels/chambers is open to the surface of the matrix to allow ingress of a liquid vaccine preparation. Filling of the holes/channels/chambers of the matrix may be accomplished by any suitable method, e.g. by dipping, soaking, painting, washing, pressing in a paste (e.g. peanut paste) containing the viral antigens, or otherwise exposing the surface of the matrix to a liquid vaccine preparation under conditions that allow or promote entry of the vaccine preparation into the holes/channels/chambers through the opening on the surface. In one embodiment, a supralingual applicator is dipped into a liquid composition containing the virus (e.g. in a cold cup), the virus enters (soaks into) the capillary holes and the supralingual applicator can then be applied to the tongue. FIG. 6A shows a top view of a matrix with channel or capillary openings 81 which open onto the surface of matrix 80. FIG. 6B depicts capillaries 82 extending within matrix 80, and connected to (opening onto) the surface via openings 81. Unlike drops, this applicator allows slow application of the vaccine on the upper surface of the tongue over several minutes.

Advantageously, in this embodiment of the invention, the matrix may be attached to a handle (which may be hinged), as shown in FIGS. 6C and D, to facilitate holding and dipping, and then administration to the recipient. As depicted, handle or holding means 90 is attached to matrix 92 which comprises channels 93, which may open on only one surface of the matrix (6C) or may extend through the matrix (6D), or a combination of both. In some embodiments, after loading of the vaccine preparation, the matrix is briefly washed or wiped to remove surface virus that is not associated with (located in) a capillary. In other embodiments, the supralingual applicator that is “loaded” with vaccine preparation may be coated or sealed with a barrier that traps the vaccine in the capillaries, e.g. with a light coating of an oil, peanut paste coat, saccharide solution, milk, etc. and the supralingual applicator is allowed to dry thereafter, and possibly to be stored (e.g. refrigerated, frozen, dessicated, etc.), prior to administration. In other embodiments, the addition of virus to the matrix is followed immediately or as soon as possible by administration to the recipient. The ends of a capillary (i.e. the portion of a capillary that is at the surface of the matrix, and which will come in direct contact with the tongue of a vaccine recipient) can be smooth or serrated. Smooth ends allow slow transfer of the virus-containing liquid, whereas serrated ends allow faster transfer of the virus-containing liquid. By controlling the capillary holes to be of substantially similar or uniform size, and by controlling the number of holes per square centimeter of matrix, allows titration of the dose of virus. For example, if the vaccine dose per puppy is 500 ul then 50 capillary holes×10 ul per hole will deliver the dose. This type of supralingual applicator delivered liquid vaccine is unique because, unlike simply squirting the vaccine into the mouth as drops, the supralingual applicator delivered vaccine is controlled in volume and provides longer contact time and slow delivery of the vaccine, and hence time for infection to occur. In contrast, when a vaccine is simply squirted in the mouth as a liquid, it is immediately ingested. Ingested vaccines generally do not tolerate exposure to gastric juices. Similarly, sublingual vaccine delivery is not as comfortable and easy to deliver as a non invasive, palatable supra lingual vaccine.

In one embodiment of the invention, the antigen that is delivered supralingually is a parvovirus from a species of wildlife such as a raccoon. A suitable raccoon parvovirus is described, for example, in Kapil et al. (Veterinary Record 2010. 166, 24-25).

### Vaccine Recipients

The supralingual vaccines described herein are used to vaccinate animals, frequently mammals (including humans), especially young mammals, and more especially neonates (newborns). In one exemplary embodiment, the mammal is a canine, e.g. a puppy, but this need not always be the case, and references to puppies or dogs herein are made for exemplary purposes as the invention can be practiced with other species of animals (e.g. other mammals, including humans). The vaccine formulations can be used to induce a mucosal immune response in any animal which is capable of licking or sucking the compositions that contain the vaccinogen. Examples of other animals species for which a supralingual vaccine is suitable include but are not limited to: felines, including domestic cats, and large cats e.g. those in captivity; ferrets; guinea pigs; livestock, e.g. cattle, sheep, goats, etc; horses; and other that will occur to those of skill in the art. With respect to administration to a neonate, the compositions may be administered to any animal that nurses soon after birth and that retains the ability to suckle for the period of time during which it is desirable to administer a vaccine. The tongue is an ideal site of immunization because it has receptive target epithelium, antigen processing cells, lymphatic and micro vasculature, lymphatics, and is surrounded by and includes strong masticatory muscles.

### Methods of the Invention

The invention provides methods for vaccinating an animal by supralingual delivery to the animal of one or more antigens as described herein. Administration of one or more antigens generally elicits an immune response in the vaccine recipient, e.g. the recipient produces antibodies and/or a cell mediated immune response to the antigens. In some embodiments, the immune response is protective, i.e. when the animal is subsequently challenged with an infectious agent containing antigens and/or antigenic determinants identical or similar to those in the vaccine composition, i.e. no disease symptoms occur in the animal, and/or disease symptoms that occur are significantly milder than those which would have occurred, had the animal not been vaccinated as described herein.

### Vaccine Regimens (Protocols)

Without the delivery system of the invention, a newborn animal (e.g. a puppy) is totally dependent on maternal immunity until a traditional vaccine regimen is initiated, e.g. at 6 weeks of age. While traditional vaccine regimens could be started at a younger age, it is not possible to accurately determine when maternal antibodies will cease to interfere with the vaccine, and when administered too early, the time and expense involved are wasted. The present invention provides a convenient, low-cost method to administer early doses of vaccine even by owners at home, before a traditional regimen is necessary. In fact, the ease and convenience of administration encourages pet owners themselves to vaccinate animals at an early age, without the need to schedule a veterinary appointment to do so.

Only 3-5% of maternal antibodies are present in new born puppies at birth. Thus, only a limited amount of antibodies circulates in a new born puppy. The period of normal colostrum antibody absorption does not generally extend beyond 24 hours after birth. (Antibody is not found in puppies' serum 24 hr after passive immunization of a pregnant bitch even though bitch titers remained high.) The greatest amount of antibody from a single feeding is absorbed around 10 hr after ingestion and absorption is almost complete at 15 hr. No antibody is detected in the serum 5 hr after feeding hyperimmune serum. The movement of large molecules through the intestinal epithelium into the vascular system is a rather slow process. Steroids promote and are involved in antibody absorption.

Puppies will thus absorb maximum amounts of antibody when a formulation of the invention is administered about 8 hr after birth, e.g. from about 2 to about 10 hours, i.e. about 2, 3, 4, 5, 6, 7, 8, 9, or 10 hours after birth. In some embodiments, to induce active immunity in a newborn puppy, the vaccine is administered directly after birth. For this type of procedure, vaccine administration is done before the maternal colostrum antibody is fed to the puppy. This process of vaccination is called simultaneous vaccine and antibody application. In other embodiments, the vaccine is delivered later (i.e. after some nursing) and directly after administering the vaccine, the puppy is encouraged to nurse again, e.g. for about 15 minutes.

In one vaccination protocol the puppy is allowed to lick the supralingual applicator and then the feeding of colostrum is not allowed for about 1 hour. Most viruses bind to the surface receptors and are internalized inside the cell within 20 minutes. Once inside the cell the virus is protected from maternal antibody neutralization. Thus, the cessation of or delay in nursing can facilitate infection of basal cells of tongue epithelium by the virus.

Puppies are born without teeth but baby canines (the longer teeth on each side in the front of the mouth) begin erupting at around 3 to 4 weeks of age, with incisors (the tiny teeth in the front) and premolars (larger side teeth) coming in at around 4 to 6 weeks of age. Puppies should have a total of 28 baby teeth by the age of 8 weeks. Generally, the vaccine formulations and delivery methods of the invention can be used for puppies of 4 weeks or less. The teething schedule in puppies further prevents biting of the supralingual vaccination devices. In some embodiments, the vaccine is administered e.g. at 2-3 weeks, followed by booster doses at 4 and 6 weeks. In some embodiments, the supralingual method is not used after 8 weeks, as traditional delivery methods (e.g. subcutaneous injection) may then be employed if needed. The need for subsequent shots can be determined serologically by determining or measuring the titer of antibodies against the antigen (e.g. CPV).

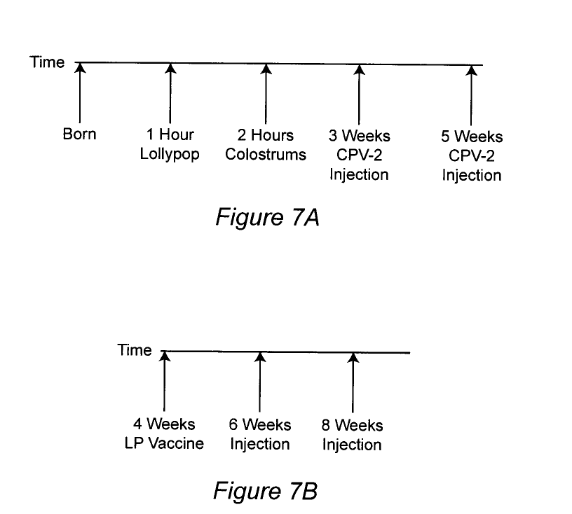

Two exemplary protocols (timelines) for vaccine administration are provided in FIGS. 7A and B, where the vaccine is first administered via supralingual applicator (LP), and later by injection. FIG. 7A shows a regimen that begins at birth; FIG. 7B shows a regimen that begins at 4 weeks of age.

Details of the procedure to apply a supralingual applicator (e.g. a lollipop) to a new born puppy: Before applying the lollipop to a new born puppy the mouth and nostrils have to be cleaned and the puppy has to be dried. When the puppy is stable (e.g. in 30 minutes), vaccinate with an applicator of the correct size as described in herein.

Immunological inertia is a phenomenon due to which a new born of any species is unable to mount a strong protective immune response to neonatal vaccines or antigens. This inertia is due to many factors that lead to requirement of puppy series of vaccination (e.g. 5 vaccine injections). In one embodiment, the use of this novel supralingual vaccination will obviate the need for puppy shots. In some embodiments, only one exposure of a new born puppy to a supralingual applicator vaccine will lead to active immunity and thus overcome immunological inertia. Puppies do have a full complement of immune system components at birth.

In an exemplary protocol for vaccine administration, the supralingual vaccine is administered to a new born puppy. After one hour colostrum is administered, e.g. by nursing. Two weeks later, a booster dose of vaccine is administered by supralingual applicator and normal feeding resumes after one hour of fasting. At 4 and 8 weeks of age, the puppy is bled and antibody titers are measured, e.g. by IFA or ELISA. Unlike the unvaccinated controls, the supralingual applicator vaccinated puppies will have IgM and IgG against the virus. This result will prove successful “take” of a vaccine that is administered by the supralingual route.

### Example 1

### Distribution of CPV in Infected Dogs

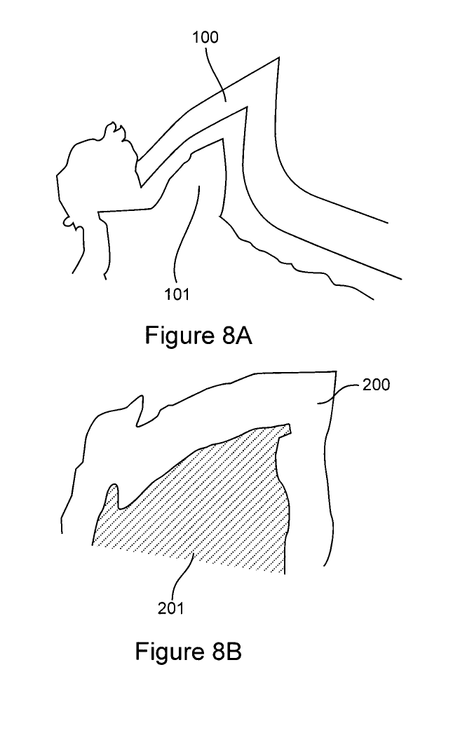

The tongue consists of a core of skeletal muscles surrounded by stratified squamous epithelium on the dorsum. The epithelium is generally thick and keratinized on the dorsum and non keratinized and thin on the ventral surface of the tongue. The dorsum of the tongue is covered with lingual papillae of two types: keratinized “filiform” papillae and non keratinized papillae. The filiform papilla, which are the most numerous, are supported by a highly vascularized connective tissue core, and are shaped like rose thorns with curvature directed caudad. Filiform papillae are numerous and well developed in both dogs and cats. Cats have filiform papillae with two prominences of unequal size (FIG. 8A). The caudad prominence is large and caudally directed. The filiform papillae of dogs have two or more apices (FIG. 8B). The caudad apex is the largest and the stratum corneum of dogs is thicker than that of the other apices. Conical papillae occur on the root of the tongue in dogs and cats. They are larger than filiform papillae and not highly keratinized.

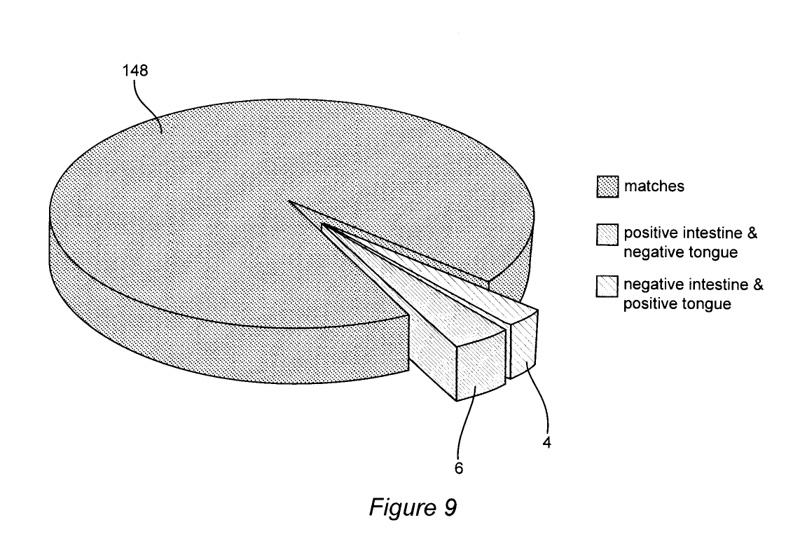

FIG. 9 shows the results of field investigations of the distribution of CPV virus in about 158 necropsies performed on dogs or puppies infected with CPV. As can be seen, co-location of CPV in both the tongue (basal cells) and the intestines occurred in nearly all CPV infected canines examined. Thus, the tongue is easily infected simply by natural application during the self cleaning procedure. Nevertheless, repeated attempts to isolate live virus from the tongue by performing virus isolation in cell lines failed, whereas attempts to isolate live virus from the intestines of the same canine succeeded. Thus, the mode of viral replication of CPV (e.g. CPV-2) in the tongue is not productive compared to replication (in the cysts of Liberkahn) in the intestine. The tongue thus allows a low level of virus persistence but not overt productive replication of CPV. While the tongue and intestines show almost 100% correlation for co-location of CPV, the level and extent of productive infection (virus titer) is significantly different.

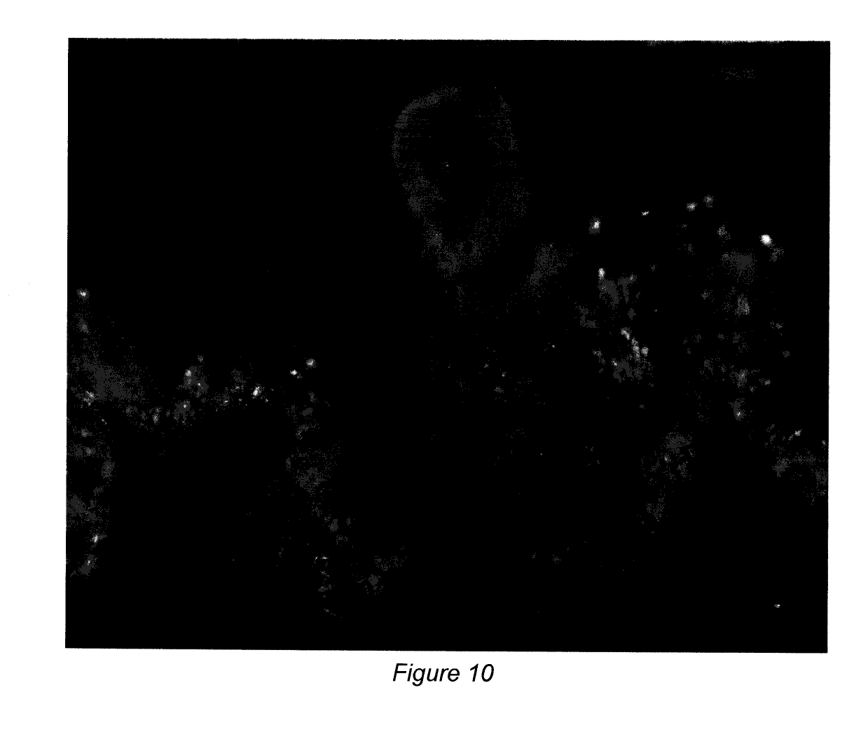

Sections of the tongue were also examined by direct fluorescent antibody testing using an anti-CPV fluorescein FITC conjugate, and FIG. 10 shows an exemplary result. It was found that only the dorsal surface of the tongue was positive for CPV antigen; the middle muscular portion of the tongue and ventral surfaces were negative. In fact, the basal layer of dorsal epithelium was infected with CPV-2 where the virus persisted for at least 1 to 2 months after the start of diarrhea symptoms due to CPV. CPV antigen was present between the filiform papillae below the epithelium in the basal dividing layer of the epithelium. In addition, some non-keratinized papillae cores with dividing cells were also infected. As described above, the filiform papillae are keratinized, and this finding shows that keratinized areas of the tongue were not infected. Rather, infection was limited to non-keratinized invaginated areas between and at the base of the papillae. The three distinct regions around the filiform papillae have been histologically defined.

This observation also explains a route of CPV auto-infection in dogs. Autoinfection likely occurs due to mechanical shear and sloughing of infected rectal epithelial cells during self-cleaning. Thus, when a dog is already infected with enteric parvovirus, the presence of the keratinized thorn-like papillae promotes deeper application of the parvovirus into the tongue, into the susceptible basal layers of the tongue, establishing CPV infection. This natural “auto infection” was observed in all positive cases of natural infection with CPV-2. Based on the distribution of the papillae, it is likely that the anterior portion of the dorsum of the tongue allows more abrasive mechanical function and the posterior dorsum of the tongue allows more absorption of virus from saliva. These same mechanisms allow delivery of a virus from the vaccine formulations of the invention to the basal cells of the tongue of an animal vaccinated as described herein.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. A method of immunizing an animal, comprising the step of
supralingually administering at least one antigen to said animal

2. The method of claim 1 wherein said at least one antigen is an attenuated virus.

3. The method of claim 2, wherein said attenuated virus infects tongue cells of said animal.

4. The method of claim 3, wherein said attenuated virus infects basal tongue cells of said animal.

5. The method of claim 2, wherein said attenuated virus is canine parvovirus (CPV).

6. The method of claim 2, wherein said attenuated virus is selected from the groups consisting of foot-and-mouth virus, feline calicivirus, feline panleukopenia virus and feline parvovirus.

7. The method of claim 1, wherein said step of supralingually administering includes presenting said at least one antigen to said animal in a formulation which includes a solid matrix with said at least one antigen dispersed in said solid matrix, and with an abrasive substance dispersed in said solid matrix.

8. The method of claim 1, wherein said step of supralingually administering is performed a plurality of times over a period of weeks.

9. The method of claim 1, wherein said animal is a neonate.

10. The method of claim 9, wherein said step of supralingually administering is performed immediately after birth of said neonate and prior to a first provision of maternal colostrum to said neonate.

11. The method of claim 9, wherein after said step of supralingually administering is performed, said neonate is not allowed to nurse for a period of time.

12. The method of claim 2 wherein said attenuated virus is genetically engineered to present one or more antigens specific for one or more infectants in said animal.

13. The method of claim 7, wherein said step of supralingually administering includes a step of contacting a tongue of said animal with said solid matrix for a period of time selected from the group consisting of 1 minute, 2 minutes, 3 minutes, 4 minute and 5 minutes.

14. An immunogenic formulation for supralingual delivery to an animal, comprising:
a matrix; and
at least one antigen dispersed in said matrix.

15. The immunogenic formulation of claim 14, wherein said matrix is a solid.

16. The immunogenic formulation of claim 14, further comprising an abrasive substance dispersed in said matrix.

17. The immunogenic formulation of claim 14, wherein said at least one antigen is an attenuated virus.

18. The immunogenic formulation of claim 17, wherein said attenuated virus infects tongue cells of said animal.

19. The immunogenic formulation of claim 17, wherein said attenuated virus infects basal tongue cells of said animal.

20. The immunogenic formulation of claim 17, wherein said attenuated virus is CPV.

21. The immunogenic formulation of claim 17, wherein said attenuated virus selected from foot-and-mouth virus, feline calicivirus, feline panleukopenia virus and feline parvovirus.

22. The immunogenic formulation of claim 17, wherein said attenuated virus is genetically engineered to present one or more antigens specific for one or more infectants in said animal.

23. A supralingual applicator for administering an immunogenic composition to a puppy, comprising:
a substrate at least including a matrix; and
at least one antigen dispersed in said matrix.

24. The supralingual applicator of claim 23, wherein said matrix is solid.

25. The supralingual applicator of claim 23, further comprising an abrasive substance dispersed in said solid matrix.

26. The supralingual applicator of claim 23, wherein said at least one antigen is an attenuated virus.

27. The supralingual applicator of claim 26, wherein said attenuated virus is selected from CPV, foot-and-mouth virus, feline calicivirus, feline panleukopenia virus and feline parvovirus.

28. The supralingual applicator of claim 23, further comprising a protective plate positioned on one surface of said matrix.
